(19) United States
(12) Patent Application Publication
(10) Pub. No.: US 2012/0264617 A1
(43) Pub. Date: Oct. 18, 2012

(54) DNA SEQUENCING EMPLOYING NANOMATERIALS

(76) Inventor: John W. PETTIT, Derwood, MD (US)

(21) Appl. No.: 13/448,044

(22) Filed: Apr. 16, 2012

Related U.S. Application Data
(60) Provisional application No. 61/475,429, filed on Apr. 14, 2011.

(51) Int. Cl. C40B 20/00; G01N 21/75; C40B 40/06; G01N 27/26; C12M 1/34

(52) U.S. Cl. 506/2; 435/6.11; 435/287.2; 506/16

(57) ABSTRACT

Charge transfer doped nanomaterials such as hydrogen terminated diamond, nanotubes, nanowires or similar nanostructures are used to create a highly sensitive pH sensor, or ion sensitive sensor to directly detect the addition of a newly incorporated nucleotide when performing DNA sequencing by synthesis. A single highly integrated chip can be made to sequence many strands of DNA in a massively parallel fashion in a short amount of time with a direct electronic readout that will bring the cost, size, power consumption of sequencing DNA to very attractive and useful levels.

### REFERENCE TO RELATED APPLICATION

The present application claims the benefit of U.S. Provisional Patent Application No.

61/475,429, filed Apr. 14, 2011, whose disclosure is hereby incorporated by reference in its entirety into the present disclosure.

### FIELD OF THE INVENTION

The present invention is directed to DNA sequencing and more particularly to DNA sequencing using a nanomaterial field effect transistor (FET).

### DESCRIPTION OF RELATED ART

We have entered an age where DNA technology and information science, enabled by the

Internet and the availability of large computing and data storage capacity at very low cost, are transforming medicine, healthcare, food, agricultural, forensic, security and numerous other aspects of life in dramatic ways. To reach this vision, a number of companies have been engaged in the development of next generation DNA sequencing instruments based on Single Molecule DNA Sequencing and have received a very large amount of investment capital in recent years. When one considers that entire nations of people, as well as crops, animals and other organisms will need to be sequenced at low cost, the demand for DNA sequencing instruments will be insatiable.

The worldwide demand for DNA based healthcare and agricultural, animal, plant and forensic needs for DNA sequences and interpretations of genomes is rising at unprecedented rates. A recent market research report said that this market will grow at a 103.5% compound annual growth rate (CAGR) from 2010 levels of about $1.3 Billion. A number of companies have been funded to enter the race to create next generation DNA sequencing instruments based on Single Molecule Sequencing. Entire nations of people are envisioned to require their DNA sequence be determined, stored and interpreted to offer health care within the next ten years.

Within the next 10 years, the demand for low cost, generally considered to be $1,000.00 USD, for a human genome sequence will be huge. There is general consensus that the demand for this type of instrument will be enormous. DNA sequencing has become a booming growth market according to an article in Forbes (“Perkin Elmer Enters DNA Sequencing Market” Jan. 24, 2011).

Single Molecule Sequencing, termed “Third Generation DNA Sequencing,” has become the new standard for DNA sequencing. Basically it involves the immobilization of a strand of DNA to be sequenced and a DNA polymerase onto a substrate and detecting the incorporation of new bases as they are added to the chain in a process called “Sequencing by Synthesis.” There are several companies developing instruments to perform single molecule sequencing and they basically us the same approach, with the main difference being in the manner in which the newly incorporated nucleotide is detected. Pyrosequencing is used by 454 Life Sciences, which incorporates the use of certain enzymes to produce a chemiluminescence signal that is optically detected when a nucleotide is added to the chain. Helicos uses a pattern of tiny holes in the substrate where the DNA is immobilized that are smaller than the wavelength of the light that is used to stimulate the fluorescence molecule attached to the nucleotide added to the chain. The fluorescence stimulation light is not able to penetrate through these holes, and only an exponentially decaying evanescent wave emerges. This has the effect of only stimulating fluorescent molecules very near the surface of the substrate where the DNA strand resides and thereby vastly decreases the background fluorescence to make the detection of the single nucleotide that has been added to the chain by detecting its fluorescence emission.

These single molecule sequencing systems generally achieve rather short read lengths on the order of a hundred to a few hundred bases, but overcome this limitation by massive parallelization where many thousands of DNA segments are sequenced simultaneously on the same substrate. Using this method, Steve Quake, founder of Helicos, has sequenced his own genome and a handful of other persons have been sequenced by single molecule sequencing technology including DNA pioneer James D. Watson. The single molecule sequencing instruments just described are rather large and costly, owing mainly to the complexity of the detection instrumentation that they require.

The person who developed the pyrosequencing technology that was sold to Roche as 454 Life Sciences, Jonathan Rothberg, developed new technology that was aimed at making single molecule sequencing much more cost effective and more highly integrated using mature semiconductor technology. His idea is to detect the released hydrogen ions that are given off when a nucleotide in incorporated into the DNA chain. This has the effect of changing the pH level in the vicinity of the immobilized strand of DNA. The new company that he founded, Ion Torrent, employs semiconductor CMOS technology to implement a massively parallel array of CMOS charge or ion sensitive transistors, ISFET, on their single molecule DNA sequencing chip. Their strategy is to use mature CMOS technology and available CMOS fabrication capabilities to greatly reduce the size and cost of single molecule DNA sequencing.

### SUMMARY OF THE INVENTION

An objective is to create single molecule DNA sequencing instruments that are very compact, field portable and low cost to make DNA sequencing available to everyone and work closely thereby enable Internet based DNA information products and services worldwide.

To achieve the above and other objects, the present invention in at least some embodiments uses charge transfer doped nanomaterials such as hydrogen terminated diamond, nanotubes, nanowires or similar nanostructures to create a highly sensitive pH sensor, or ion sensitive sensor to directly detect the addition of a newly incorporated nucleotide when performing DNA sequencing by synthesis. A single highly integrated chip can be made to sequence many strands of DNA in a massively parallel fashion in a short amount of time with a direct electronic readout that will bring the cost, size, and power consumption of sequencing DNA to very attractive and useful levels.

The value products based on the present invention to the end user, the individuals who shall receive unprecedented levels of health care, is so large as to be inestimable. Governments of nations, health care providers, drug and pharmaceutical companies and biomedical institutions worldwide will seek our products and services to offer medical care and accelerate their ability to meet the needs of their nation's population or customers.

The present inventor recognized the significance of the main thrust of Ion Torrent's approach to make single molecule sequencing more cost effective by detecting the incorporated nucleotide by direct electronic means. This allows the instrument to be reduced in size by orders of magnitude as well as be more robust, reliable and energy efficient. The present inventor is heading in a similar direction, but is using the significant advantages of nanotechnology to achieve the direct detection of the incorporated nucleotide. The present inventor's approach is to use a massively parallel array of embedded nanomaterial ion sensitive sensors, including thin film carbon nanotube ion sensitive field effect transistors and hydrogen terminated diamond sensors, to detect the newly incorporated nucleotide. Carbon nanotubes are exquisitely sensitive to charge near their surface, and the present inventor has developed a number of highly effective sensors based on this principle. The present inventor has spent considerable effort in recent years developing the carbon nanotube processing technology to create uniform dispersion of carbon nanotubes in appropriate solvents so that thin film devices can be fabricated.

The present inventor has been actively involved with nanotechnology and the related field of microfluidics since the early 2000's. The present inventor's initial aim was to develop nanotechnology based industrial sensors and measuring devices for on-line manufacturing control. In 2003 the present inventor was awarded a small business SBIR contract from Air Force Research Laboratories (AFRL) to pursue carbon nanotube based passive optical sensors and then to develop optically controlled electrical switching devices for the Air Force's More Electric Aircraft and EMI Immune Control System research initiatives.

When a new nucleotide is added to a strand of DNA being synthesized by a polymerase acting together with a DNA template, a hydrogen ion is released in the formation of the phospho-diester bond. This released hydrogen ion or proton has the effect of altering the pH of the fluid in the vicinity of the DNA molecule. If the fluid in the vicinity of the DNA strand being synthesized contains only one type of nucleotide, then the detection of this hydrogen ion can be used to determine that this nucleotide has been added to the DNA strand. By washing away the existing nucleotides of a given type, one of A, C, G or T, and then bringing new nucleotides of a different type, one can measure which type of nucleotide is being incorporated in sequence to the DNA strand and therefore determine the nucleotide sequence of the DNA strand. This is the general process of sequencing by synthesis.

The technology disclosed by Rothberg in US Patent Applications US20090026082 A1 dated Jan. 29, 2009 and US20090127589 A1 dated May 29, 2009 teach the use of massively parallel arrays of silicon CMOS ion sensitive FET' s for direct detection of the released hydrogen ion and readout of many strands of DNA being sequenced in parallel. Rothberg's patents disclose that the technology of reading DNA hybridization and nucleotide incorporation by detecting the released hydrogen ion have been disclosed in the literature. Research articles published in the open literature that teach this include:

1. “Fully electronic DNA hybridization detection by a standard CMOS biochip,” Massimo Barbaro et. al., Sensors and Actuators B Chemical 118, (2006) Pgs 41-46

2. “Real Time Monitoring of DNA polymerase reactions by a micro ISFET pH sensor,” Anal. Chem. 64(17) 1992 pp 1996-1997.

A key consideration is the ultra high sensitivity needed to reliably detect the extraordinarily small change in the pH caused by the release of a single hydrogen ion. According to the theory of the pH FET or ion sensitive FET, ISFET, originally developed by Bergveld and described in the article “Thirty Years of ISFETOLOGY: what happened in the past 30 years and what may happen in the next 30 years,” P. Bergveld, Sens. Actuators, 88 (2003) pp 1-20, the limit of detection of such CMOS structures is limited by the Nerst equation to about 50 millivolts per pH unit and is in practice much less.

This limitation makes it very difficult to detect the incorporation of the nucleotide in the Rothberg approach and as a result very short read lengths with many errors are obtained. These limitations are partially overcome in the Rothberg approach by massive parallelization where more than one million strands of DNA are sequenced and results are compared and interpreted to eliminate errors. Nonetheless, there is still a great need to have accurate DNA sequencing with longer read lengths so that the massive parallelization is used to increase the throughput of the overall process.

An objective of the present invention is to improve upon the limitations of the Rothberg patent by employing ion sensitive or pH sensitive detectors that have a much better sensitivity than the silicon CMOS FETs taught by Rothberg. The present claimed invention in at least some embodiments uses nanomaterial based FETs used in the manner of ion sensitive FETs or pH sensitive FET that have much better sensitivity owing to their small size, where greater surface area to volume is realized and surface effects bring about greater sensitivity to charge. Nanomaterial based approaches have not been taught or disclosed by Rothberg or any published reference for the purpose of sequencing DNA by synthesis, but they have been disclosed for the purpose of DNA hybridization detection and bio-detection in general.

A further refinement that we teach herein is the use of charge transfer doping of the nanomaterial FET. Charge transfer doping has been found to make ion sensitive FETs from diamond that surpass the Nernst equation limitation of CMOS based FETs by a large factor. In principle ion sensitive FETs made through the use of charge transfer doping can be ten times more sensitive than silicon CMOS ion sensitive FETs. The articles “pH Sensors Based on Hydrogen Terminated Diamond Surfaces,” Jose A. Garrido et al., Applied Physics Letters, 86, 073504 (2005) and “DNA Sensors with Diamond as a Promising Alternative Transducer Material,” Veronique Vermeeren et al. Sensors 2009, 9, 5600-5636, disclose the use of charge transfer doping in hydrogen terminated diamond. Garrido et al. show data in FIG. 3 of their article where charge transfer doped diamond pH sensors produced 75 millivolt per unit of pH change, which clearly exceeds the 50 millivolts per unit pH change theoretical limit of silicon CMOS based pH sensors.

In charge transfer doping of hydrogen terminated diamond, a charge accumulation layer is induced by the electrolyte that is over the diamond surface to create a conduction layer just below the diamond's surface. A further advantage of this is that no isolation layer is needed as in a CMOS FET, so the device is much simpler to construct.

We further teach that carbon nanotube based FET' s can be used to create the ion sensitive or pH sensitive detector for use in DNA sequencing by synthesis. The highest figure of merit FET has been made with a carbon nanotube FET and reported by Fuhrer et al. It is also known that charge transfer doping of carbon nanotubes is also possible through the use, for example, of certain atoms or molecules adsorbed on the surface of the carbon nanotubes.

Other nanomaterial structures including nanowires and nanorods or quantum dots may also be employed that work by similar means through small size, confinement effects and large surface area to volume considerations.

The preferred embodiment involves the use of nanomaterials, particularly semiconducting single walled carbon nanotubes, but also single crystal diamond, nanowires and nanorods and other nanostructures that use containment and fundamental particle (electron) boundary condition effects to bring about properties that are not seen at the macro level.

Nanomaterial and nanotechnology based approaches to DNA sequencing are being announced and represent the next wave of dramatic advancement in DNA sequencing technology. Recently a company named NanoPore has announced a DNA sequencing device the size of a USB memory stick. This technology uses nano sized pores in a material that only DNA of a certain length can pass through.

The basic process of taking a biological sample, extracting the DNA, cutting the DNA up into many short segments and then sequencing these individual segments in a parallel fashion is known. Companies such as Helicos, Pacific Bioscience, Illumina, Life Technologies are doing this.

Also known in the art is the ability to sequence DNA on a single silicon chip using the principle that a proton is releases upon the incorporation of a new nucleotide when performing “sequence by synthesis.”

The present claimed invention in at least some embodiments improves upon the silicon CMOS technology used by Ion Torrent to make the fundamental measurement of pH change that accompanies the incorporation of a new nucleotide onto the DNA chain.

The silicon CMOS approach is limited by the Nernst equation to a value of 59 millivolts per unit pH change at room temperature. Since the pH change is very small upon the release of a proton upon nucleotide incorporation, a more sensitive approach is valuable.

The present claimed invention in at least some embodiments uses a nanomaterial such as carbon nanotubes to create an ion sensitive field effect transistor, ISFET, in conjunction with a charge transport layer that allows the charge, a proton in this case, to act upon the carbon nanotubes. This charge will change the Fermi level of the nanotubes and either increase or decrease the number of electrons in the conduction band. This will change the carbon nanotube's resistance, which can be measured by electrodes at either end of the carbon nanotube channel, or by optical means where the strength of the optical absorption line is modulated by the change in the number of electrons in the conduction and valence bands brought about by the Fermi level change caused by the charge acting on the carbon nanotube channel.

Ion sensitive and pH sensors, ISFET, have been made using carbon nanotubes. See accompanying review of this for biological applications. It has been disclosed in the literature the use of such ISFET for measuring the hybridization of DNA immobilized on the ISFET channel.

Nobody has disclosed the use of a carbon nanotube or other nanomaterial based ISFET for the measurement of the proton released upon the incorporation of a new nucleotide by the phospo-diester bond on the backbone of the DNA. The DNA does not need to be immobilized on the ISFET channel for this proton detection to be made, whereas the detection of hybridization needs the DNA to be connected to the channel for the change in overall charge state brought about by the hybridization to be measured by the ISFET.

The preferred embodiment uses DNA immobilized on the channel, but this is done only to bring the DNA in close proximity to the ISFET channel. DNA can alternatively be bound to beads in a process similar to what Ion Torrent discloses in the accompanying article. These DNA bound beads can be brought close to the carbon nanotube ISFET by allowing them to drop into a shallow well that is constructed around the ISFET as disclosed by Ion Torrent. This approach would work equally well with the present invention as the free proton traverses the ion transport layer to then bring about the charge effect on the carbon nanotube channel.

pH sensitive transistors have been made using carbon nanotubes, single crystal diamond and other nanostructures where charge transfer doping type of effects bring about the conductivity change in the conducting channel. Under these conditions, the change in conductivity per unit change in pH can be 10 or more times greater than the limit placed on silicon methods by the Nernst equation.

This is a huge advantage, as it allows more sensitivity, potentially greater DNA sequence reads, which are limited to 100-200 base pairs presently, although claims by various manufacturers to go to 1000 have been made, more reliability and greater assurance of accuracy in base calling and more flexibility in devising various measurement techniques and device designs.

The present invention can use optical detection of the change in pH that is simple and low cost. Ion Torrent specifically says non-optical in their approach. Ion Torrent's dislike of optical methods is their very high cost because these optical methods are implemented by laser induced fluorescence techniques. The present invention can use a much simpler method of optical transmission measurement at the carbon nanotube's resonant optical wavelength. This can be implemented with a CCD array camera to measure the optical absorption change in the many million sequencing sites on the chip. Such CCD cameras are very inexpensive and are used for instance on every cell phone.

The use of carbon nanotube and other nanomaterials based approaches to this objective of achieving highly integrated, massively parallel processing of DNA sequencing is the huge cost advantage potential of this new technology. Liquid or solution based processing can be employed where the carbon nanotubes or other nanomaterial is handled in a liquid state, deposited onto the chip in the desired pattern, and then allowed to dry or have its features created by laser ablation techniques, etching or other more economical methods that do not require the costly infrastructure that Ion Torrent mentions regarding silicon CMOS processes.

Liquid phase processing will allow DNA sensors to be placed on flexible substrates that will permit very low cost diagnostic products that can even be human wearable or disposable.

Reference Ion Torrent article:

Jonathan M. Rothberg et. al. “An integrated semiconductor device enabling non-optical genome sequencing,” Nature, Volume 475, Jul. 21, 2011, Pages 348-352

### BRIEF DESCRIPTION OF THE DRAWINGS

A preferred embodiment of the present invention will be set forth in detail with reference to the drawings, in which:

FIG. 1 shows a segment of a DNA double helix;

FIG. 2 shows a plan view of a DNA sequencing chip;

FIG. 3 shows a plan view of a single DNA sequencing site;

FIG. 4 shows one rung of a DNA ladder;

FIG. 5 shows a carbon-nanotube field-effect transistor (FET);

FIG. 6 shows a carbon-nanotube FET with a strand of DNA immobilized thereon;

FIGS. 7 and 8 show DNA sequencing on a nanotube FET;

FIG. 9 shows the binding of carbon atoms in the sequencing of FIGS. 7 and 8;

FIG. 10 shows a density-of-states diagram;

FIG. 11 shows a microfluidic chip;

FIG. 12 shows an experimental setup;

FIG. 13 shows graphs of optical absorbance;

FIG. 14 shows multiple ISFET' s; and

FIG. 15 shows cloud computing as used in the preferred embodiment.

### DETAILED DESCRIPTION OF THE PREFERRED EMBODIMENT

A preferred embodiment will be set forth with reference to the drawings, in which like reference numerals refer to like elements or steps throughout.

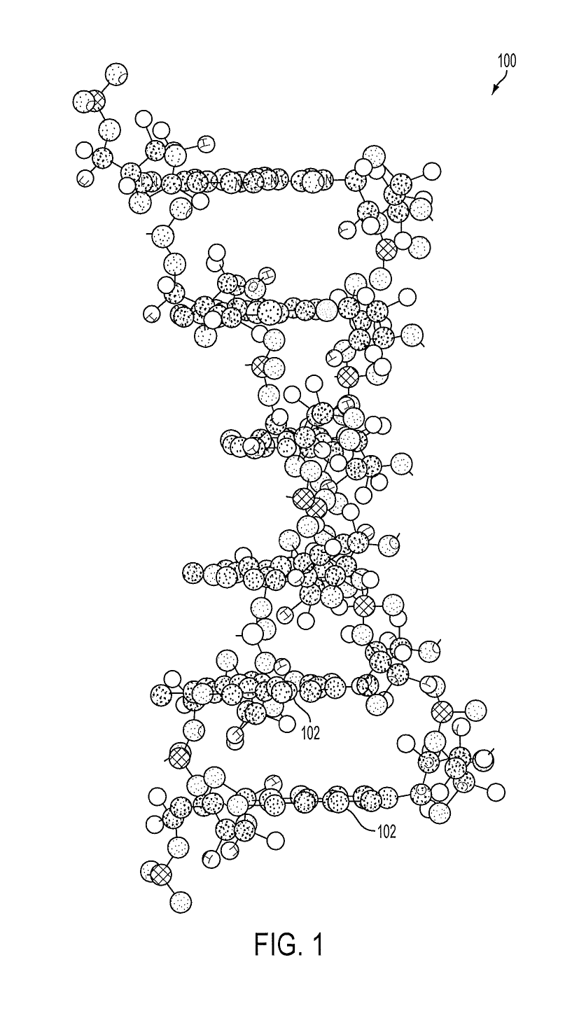

The preferred embodiment will be disclosed with reference to computer-generated 3-D models of DNA. FIG. 1 shows the DNA double helix 100—a short stretch of double-stranded DNA. Nucleotides, or “bases” combined in complimentary pairs comprise the “rungs” 102 of the double helix. All genetic information is contained in the sequence that these bases are found in any given specimen of DNA. The four DNA bases are Adenine, Thymine, Guanine, and Cytosine, abbreviated A, T, G and C respectively.

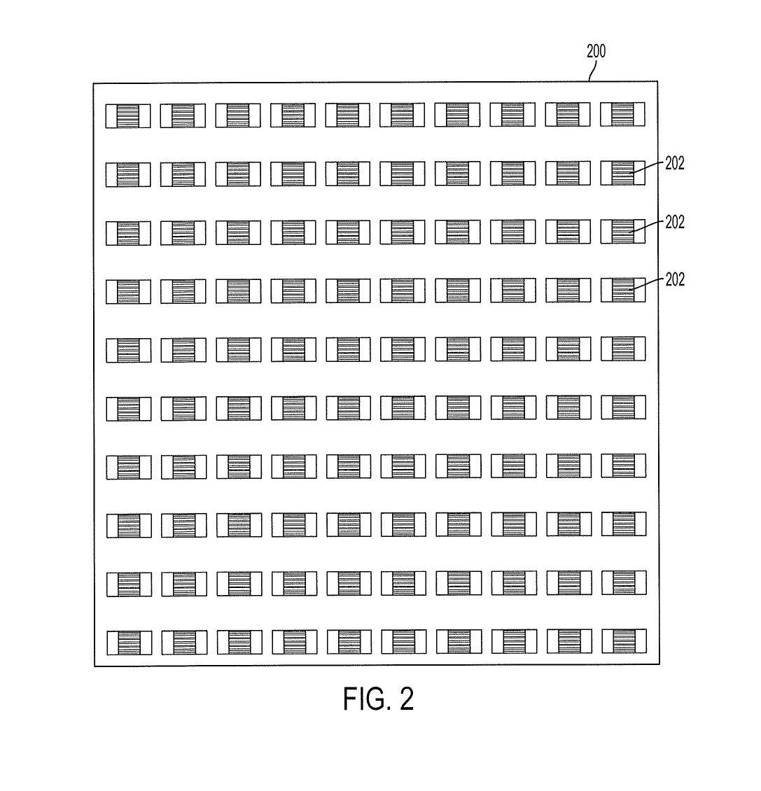

The preferred embodiment uses many DNA sequencing sites 202 fabricated on a single chip 200 with solution phase carbon nanotube technology which is low cost, small in size and very economical to manufacture. FIG. 2 shows a plan view of a DNA sequencing chip. The preferred embodiment uses massively parallel arrays of DNA sequencing sites on a single chip, with 8 million or more DNA sequencing sites working in parallel.

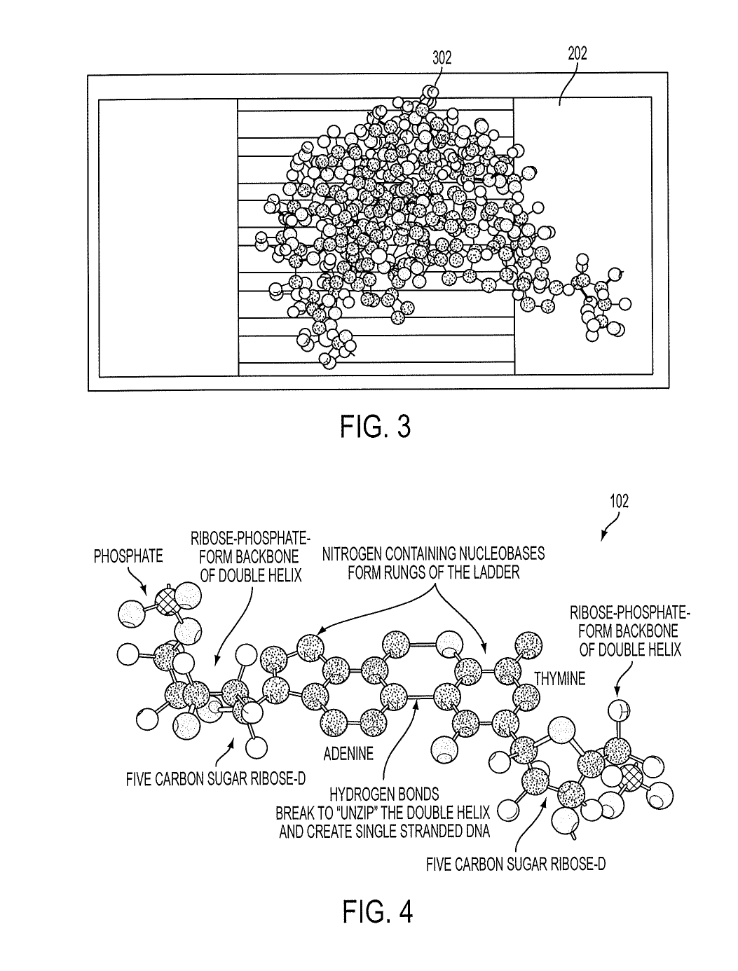

FIG. 3 shows a plan view of a single DNA sequencing site 202. Single stranded probe DNA 302 is immobilized onto the detection channel. The signal received when a new nucleotide base is added is used to determine the next base in the DNA sequence.

Each sequencing site would in many practical applications have many identical copies of the single stranded DNA either immobilized on the channel or brought into close proximity by techniques such as binding the many copies of the single stranded onto a bead that is then brought to the channel and contained in a shallow well with approximate dimensions of 1 to 4 microns on a side.

FIG. 4 shows one rung 102 of the DNA ladder showing an A-T link, namely, Adenine-Thymine linked by relatively weak hydrogen bonds forming the “Rung” of the ladder, and also shows the five carbon sugar and phosphate that create the DNA backbone This forms one “rung” of the DNA double helix

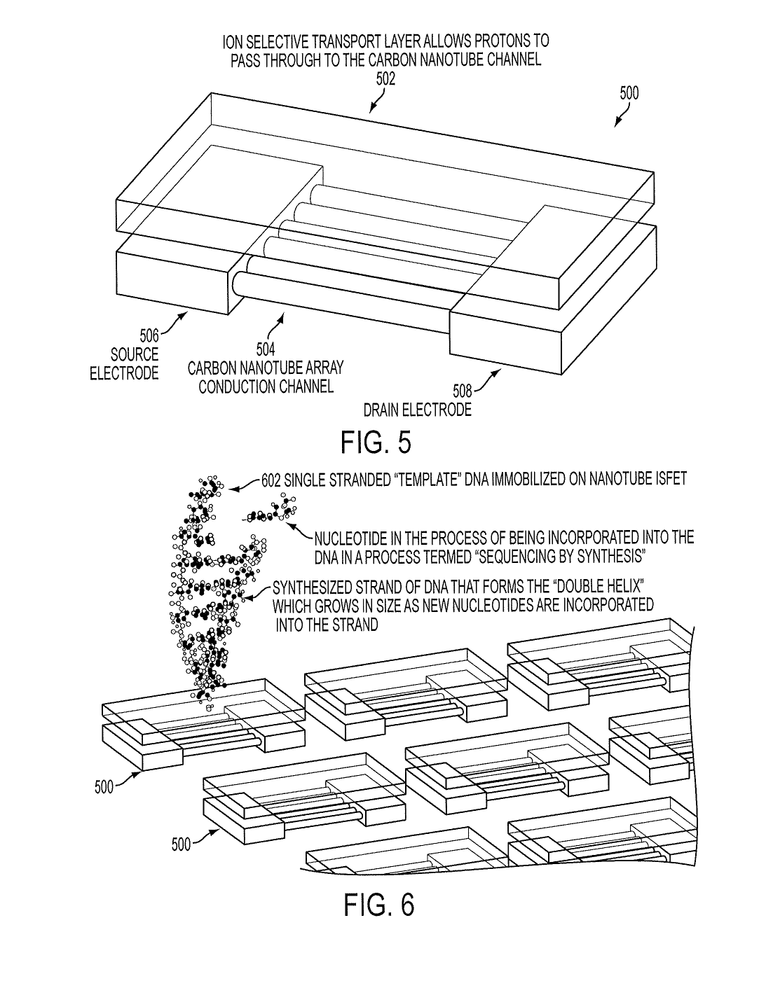

The preferred embodiment, at each site 202, uses an ion selective carbon nanotube field effect transistor or ISFET, shown in FIG. 5 as 500. An ion transport layer allows the proton in this case travel through to the carbon nanotube channel and create a small voltage that can be detected across the carbon nanotubes 504 at electrodes labeled Source 506 and Drain 508.

Protons released when the phospho-diester bond is formed upon incorporation of a new nucleotide are transported to the surface of the carbon nanotubes. Through charge transfer doping effects this creates a signal that is more than ten times greater than possible with CMOS silicon transistors.

A single stranded “Template” segment of DNA is attached to the ISFET over the channel. This template's sequence is to be determined by seeing which base in sequence successfully incorporates itself to the template to build the “double helix.” When a new base is incorporated, a proton is ejected that is detected by the ISFET. The ISFET acts like a small pH sensor.

Although each site is shown with only one single stranded DNA template attached to it, each sequencing site would in many practical applications have many identical copies of the single stranded DNA either immobilized on the channel or brought into close proximity by techniques such as binding the many copies of the single stranded onto a bead that is then brought to the channel and contained in a shallow well with approximate dimensions of 1 to 4 microns on a side.

Each Nanotube ISFET 500 has a segment of single stranded “Template” DNA 602 immobilized on it, as shown in FIG. 6. Several million such structures act together to sequence the DNA in a massively parallel fashion.

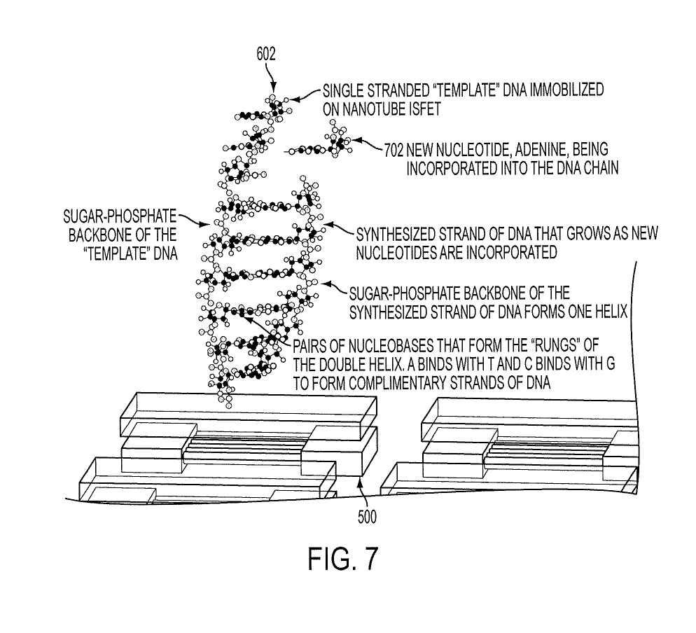

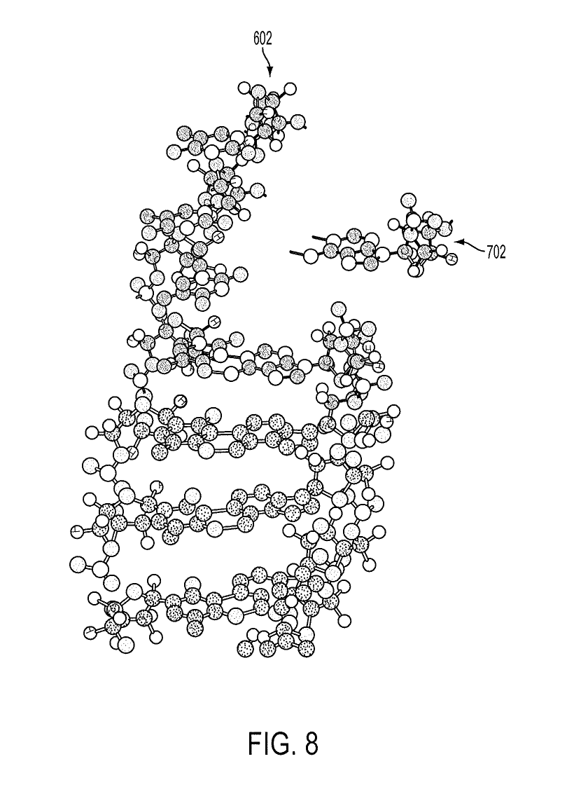

DNA sequencing on a nanotube ISFET is shown in FIGS. 7 and 8. A nucleotide, Adenine in this example, is being incorporated into the DNA. A proton is released upon the formation of the Phospho-Diester bond when the Nucleotide is incorporated. This proton is then detected by the Nanotube ISFET indicating that Adenine is the next base in this sequence. This proton release is equivalent to a drop in the pH level of the fluid in the proximity of the DNA. The ISFET responds to this pH change.

Alternatively, this pH change can be detected by a low cost optical approach where a light beam passes through the channel and the optical absorption of the channel at the carbon nanotube's resonant frequency is measured.

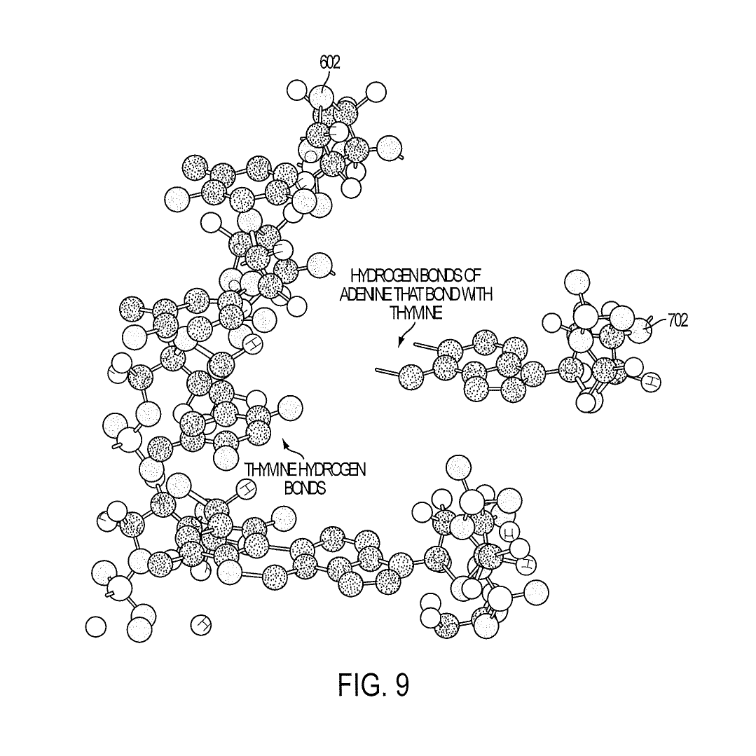

FIG. 9 shows the number 3 carbon of the new nucleotide binds with the number 5 carbon of the last nucleotide in the existing strand, forming a Phospho-Diester bond and releasing a proton.

A proof-of-principle experiment will now be disclosed. A microfluidic chip was fabricated by forming a layer of semiconducting carbon nanotubes at the bottom of the chamber of the chip. Over this layer of carbon nanotubes, an ion transport layer was added. In this case this layer was Polyethylene Oxide (PEO) with Lithium Perchlorate (LiClO₄) where the Lithium ions are solvated by the PEO in a manner that is known in the art. Water of various pH values was then allowed to flow over this chamber. An optical detection probe was used to measure the change in absorbance of this carbon nanotube film at the resonant wavelength of the carbon nanotubes at the V1 to C1 transition. This wavelength for the carbon nanotubes used in this experiment was at approximately 1020 nanometers.

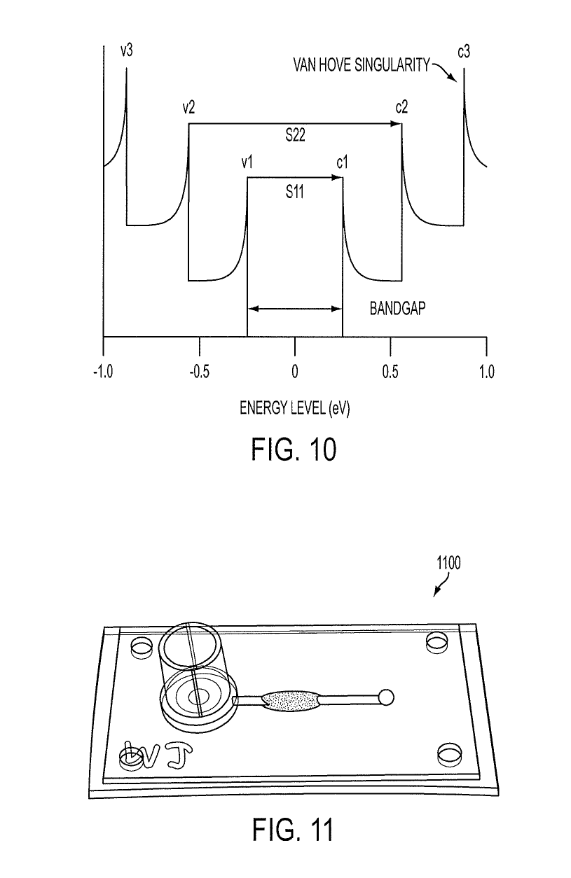

FIG. 10 shows a Semiconducting Carbon Nanotube Density of States (DOS) Diagram showing allowed energy levels for electrons in the valence band (v1, v2, v3) squeezed into van Hove Singularities, the forbidden bandgap and electrons at allowed energy levels in the conduction band (c1, c2, c3). A carbon nanotube can absorb the energy of an optical photon when the ephoton's energy corresponds to the energy difference from a van Hove singularity in the valence band to a van Hove singularity in the conduction band. Transitions for the first pair are termed S11 transitions and the second pair are termed S22 as shown in FIG. 10.

Modulate the strength of the direct bandgap transition in semiconducting nanotube by shifting the Fermi level through charge acting on the nanotube's surface.

Choose nanotubes with narrow diameter and hence bandgap distribution that is at workable value for fiber optic networks.

SouthWest Nanotechnologies has nanotubes with ˜1 eV bandgap yielding a v1 to c1 transition at ˜1020 nanometers.

The design of the microfluidic chip will now be described. As shown in FIG. 11, a simple microfluidic chip 1100 was fabricated that has a chamber with approximate dimensions of 2 millimeters wide by 4 millimeters long and 200 microns deep. A carbon nanotube layer was placed on the bottom of this chamber and an overlayer of Polyethylene Oxide (PEO) mixed with Lithium Perchlorate (LiClO₄) was placed over the carbon nanotube layer to create the ion transport layer. Water with various pH values was then allowed to flow over this chamber from the cylindrical fixture at the left side through the visible microfluidic channels then over the chamber and then expelled at the opening on the right.

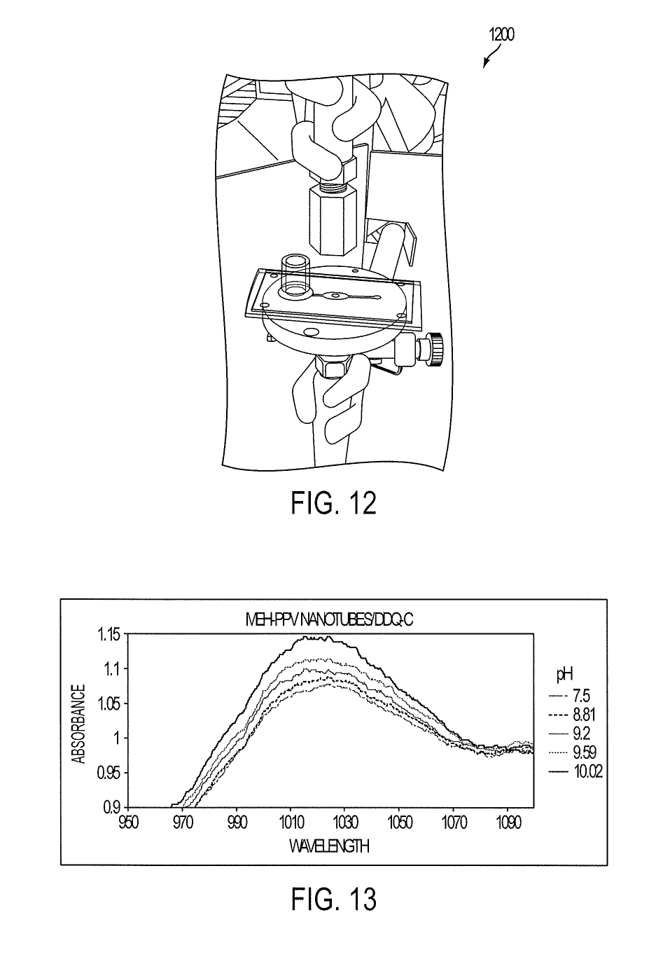

FIG. 12 shows the experimental setup 1200. An optical fiber brings a beam of light from above onto the chamber with carbon nanotube PEO LiClO₄ composite. The transmitted light is received by the optical fiber at the bottom. This light is then brought to an optical spectrometer for analysis that is shown next.

Results of the above experiment will now be disclosed. FIG. 13 shows the optical absorbance of carbon nanotube film versus pH value of water in chamber. Changes in the optical absorption of the carbon nanotube film as detected by the optical probe setup shown above. Water with various pH values was flowed through this chip over the chamber and the optical spectra was recorded. FIG. 13 shows a clear response to various pH values at the resonant optical wavelength of 1020 nanometers.

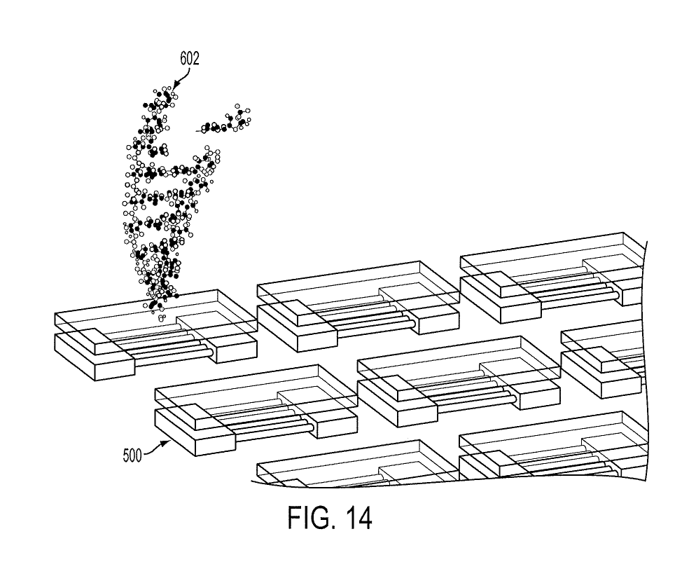

As shown in FIG. 14, each Nanotube ISFET has a segment of single stranded “Template” DNA immobilized on it. Although each site is shown with only one single stranded DNA template attached to it for clarity in presentation, each sequencing site would in many practical applications have many identical copies (up to many millions) of the single stranded DNA either immobilized on the channel or brought into close proximity by techniques such as binding the many copies of the single stranded onto a bead that is then brought to the channel and contained in a shallow well with approximate dimensions of 1 to 4 microns on a side.

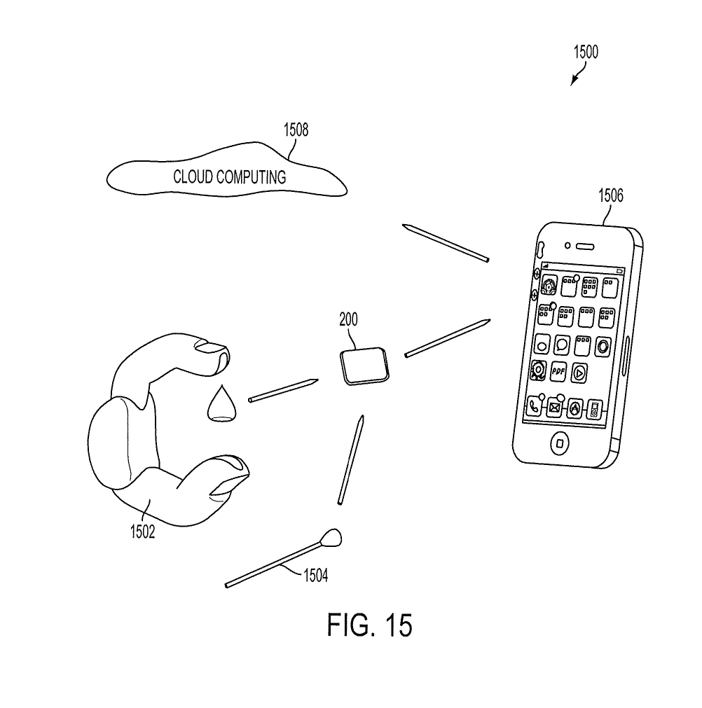

Several million such structures act together to sequence the DNA in a massively parallel fashion. The many million individual short strand DNA sequences need to be matched together end to end to form the overall DNA sequence. This process is rather computing intensive. One concept is to use Cloud Computing where the millions of individual short strand DNA sequences generated by a single chip are transmitted wirelessly by cellular technology to the Internet or “Cloud” as it is being called, to perform these computations to determine the overall sequence and other analyses of the DNA. As shown in FIG. 15, in a cloud computing environment 1500, a DNA sample to be sequenced is brought to the chip 200 as a drop of blood 1502, buccal swab 1504, or any similar method of collecting a DNA sample. The chip 200 then makes many million individual DNA sequences from the DNA by first cutting the DNA into many short segments using methods that are well known in the art. The chip, or the electronics that accompany the chip, then transmits these individual DNA segment sequences over a smartphone 1506 or any other suitable device to the “Cloud” 1508 for processing. Wireless technology is a preferred method, but wired connections such as through a USB bus connection are possible.

While a preferred embodiment has been disclosed in detail above, those skilled in the art who have reviewed the present disclosure will readily appreciate that other embodiments can be realized within the scope of the invention. For example, numerical values are illustrative rather than limiting, as are disclosures of specific technologies. Therefore, the present invention should be construed as limited only by the appended claims.

I claim:

1. A method for sequencing DNA, the method comprising:
(a) causing the DNA to react with probe DNA adjacent to a nanomaterial ion sensitive or pH sensitive field-effect transistor (FET) to emit a hydrogen ion;
(b) detecting the hydrogen ion through use of the nanomaterial ion sensitive or pH sensitive FET; and
(c) sequencing the DNA in accordance with a result of step (b).

2. The method of claim 1, wherein the nanomaterial is hydrogen terminated diamond.

3. The method of claim 1, wherein the nanomaterial is carbon nanotubes.

4. The method of claim 1, wherein the FET is made by use of charge transfer doping of the nanomaterial.

5. The method of claim 1, comprising massively parallel DNA sequencing by means of an array of said FET's.

6. The method of claim 1, wherein step (b) comprises optically detecting a change in pH.

7. The method of claim 1, wherein step (b) comprises detecting a voltage across the FET.

8. The method of claim 1, wherein step (c) comprises cloud computing.

9. A chip for sequencing DNA, the chip comprising:
a nanomaterial ion sensitive or pH sensitive field-effect transistor (FET); and
probe DNA immobilized adjacent to the FET.

10. The chip of claim 9, wherein the nanomaterial is hydrogen terminated diamond.

11. The chip of claim 9, wherein the nanomaterial is carbon nanotubes.

12. The chip of claim 9, wherein the FET is made by use of charge transfer doping of the nanomaterial.

13. The chip of claim 9, comprising an array of said FET's for massively parallel DNA sequencing.
